(19) United States
(12) Patent Application Publication
(10) Pub. No.: US 2004/0042995 A1
(43) Pub. Date: Mar. 4, 2004

(54) Use of methyl methacrylate polymers as texturing agents in cosmetic or pharmaceutical compositions, and compositions containing them

(76) Inventors: Alicia Roso, Saix (FR); Catherine Bulcourt, Boulogne Billancourt (FR); Nelly Michel, Maisons Alfort (FR)

Correspondence Address: Linda K. Russell, Air Liquide, 2700 Post Oak Blvd., Suite 1800, Houston, TX, 77056, US

(21) Appl. No.: 10/457,674

(22) Filed: Jun. 9, 2003

(30) Foreign Application Priority Data
Jun. 12, 2002 (FR) 0207198

(51) Int. Cl. A61K 7/06; A61K 7/11

(52) U.S. Cl. 424/70.16

(57) ABSTRACT

Use of a polymer or of a blend of polymers chosen from copolymers or terpolymers of methyl methacrylate with one or more monomers chosen from acrylic acid esters or methacrylic acid esters, as a texturing agent in cosmetic or pharmaceutical compositions formulated in the form of powders that may be applied to the human or animal body. Cosmetic or pharmaceutical composition containing it, and use thereof as makeup products.

The invention relates to the use of polymer powders to prepare cosmetic compositions more particularly intended for making up the skin.

Skin makeup compositions generally contain texturing agents consisting of spherical or lamellar grains of powder to make them soft on application to the skin. However, these powders are difficult to use in the preparation of pressed powders, which is one of the cosmetic formulations most commonly used for these makeup compositions.

Specifically, these texturing agents are “incompactable” solid fillers, i.e. they do not allow satisfactory compacting to be achieved in standard compression processes. The term “satisfactory compacting” means a pressed powder having a smooth, flat surface and retaining its compactness over time while at the same time having a sufficient impact strength.

With such “incompactable” fillers, starts to fragment and disintegrate the pressed powder is observed as soon as the amount used exceeds 1% by weight of the pressed powder on account of the appearance of a phenomenon of relaxation of the particles.

Examples of “incompactable” fillers include:

Polymethyl methacrylates, often denoted in the literature as PMMA, and which are formed from microporous microspheres with a specific surface area of greater than or equal to 0.5 m² per gram, such as those sold under the names Micropearl™ M305 and Micropearl™ M100;

Silica microspheres, such as those sold under the name Silicabeads™ or Polytrap™;

Hollow microspheres made of thermoplastic material, for instance, polyethylenes, polystyrenes, polyacrylonitriles or polyamides, such as those sold under the names Orgasol™, or alternatively made of polyesters, such as those sold under the name Expancel™;

Microcapsules made of organic or mineral material, such as those sold under the name Macrolite™; or

Certain lamellar fillers, such as Sericite BC282™ and talc Extra Steamicoos™.

At the present time, it is not known how to incorporate more than 1% by weight of such texturing agents into a makeup formulation, other than by performing a particular manufacturing process including an extrusion step. Such a process is described in the European patent application published under No. EP 0 651 991 A1. This extrusion step involving the use of specific apparatus thus limits the use of such fillers, especially in the small-scale and medium-scale cosmetics industry which generally has available only standard compression means.

Accordingly, the Applicant set itself the task of investigating novel texturing agents that do not have the drawbacks presented above. It has found that, contrary to all expectation, certain methyl methacrylate copolymers, by virtue of their suppleness, their intrinsic capacity to be deformed and their greater elasticity, can be more effectively compressed, by promoting the step of plastic deformation required for good cohesion of their powder of spherical particles and by reducing the relaxation phenomenon at the end of compression, the said phenomenon being responsible for the disintegration and fragmentation effects of the pressed powder obtained.

Consequently, one subject of the invention is the use of a polymer or a blend of polymers chosen from copolymers or terpolymers of methyl methacrylate with one or more monomers chosen from butyl acrylate, 1-methylpropyl acrylate, 2-methylpropyl acrylate, 1,1-dimethylethyl acrylate, butyl methacrylate, 1-methylpropyl methacrylate, 2-methylpropyl methacrylate and 1,1-dimethylethyl methacrylate, as texturing agent in cosmetic or pharmaceutical compositions formulated in powder form, which may be applied to the human or animal body.

In the context of the present invention, the term “powder” means that it is either a powder dispersed in an aqueous or organic phase such as a wax, an oil, an organic solvent or a mixture of these phases or in a emulsion of water-in-oil, oil-in-water or multiple type, or alternatively in a solidified anhydrous medium, for instance a mascara, a nail vanish, a fluid foundation, a lipstick or a foundation stick, or a powder, whether it is in free or compacted form, for instance free or pressed powders for unifying the complexion, talcs or body powders, foundation cakes, blushers and eye shadows.

The polymers used, as defined above, are known and their preparation is described, for example, in European patent application EP 1 041 095. They are in the form of microspheres.

A subject of the invention is more particularly the use as defined above, in which the cosmetic or pharmaceutical compositions are pressed powders.

In the context of the present invention, the term “pressed powder” means a compacted tablet obtained by mechanical compression of the initial powder mixture, the said compact state needing to be maintained over time, the said tablet needing to have a flat and smooth surface, to have a sufficient impact strength so as not to break, and the constituent powder of which may be taken up easily using a finger, a foam applicator or a brush, to be applied to the skin.

According to a first particular aspect of the present invention, a subject of this invention is the use, as defined above, of a copolymer of methyl methacrylate with one or other of the monomers chosen from butyl acrylate, 1-methylpropyl acrylate, 2-methylpropyl acrylate and 1,1-dimethylethyl acrylate.

According to a most particular embodiment of the present invention, it concerns the use, as defined above, of a copolymer of methyl methacrylate and of butyl acrylate.

Polymers of this most particular type are sold under the name Microsphere™.

A subject of the invention is also a cosmetic or pharmaceutical composition formulated in the form of a powder, which may be applied to the human or animal body, characterized in that it comprises:

an effective amount of a polymer or of a blend of polymers as defined above, and

at least one cosmetically acceptable compound.

In the context of the present invention, the expression “at least one cosmetically acceptable compound” means at least one compound chosen especially from dyes, pigments, hydrophobic binders, for instance fatty acid esters, waxes, mineral, plant or synthetic fillers, UV-A or UV-B sunscreens, fragrances, and fixing agents therefor, preserving agents and texturing agents.

As pigments, there are more particularly pigments that may be used in cosmetic or pharmaceutical formulations intended to be applied to the skin, for instance titanium dioxide, bismuth oxychloride, brown iron oxides, yellow iron oxides, black iron oxides or red iron oxides, or alternatively white or coloured nacreous pigments such as titanium mica.

Examples of waxes include beeswax, ozokerite, carnauba wax and candelilla wax.

Examples of fillers include microcrystalline cellulose, methylcellulose, kaolin, talc, micas, starch, for instance rice starch, magnesium carbonate, magnesium hydrocarbonate, silica, polyethylene derivatives, calcium carbonate, zinc oxide or titanium oxide or metal salts of fatty acids.

Examples of fatty acid esters include the compounds of formula (I):

R₁—(C═O)—O—[[CH₂—CH[O—[C(═O)]ₘ—R₂]—CH₂—O]ₙ—[C(═O)]ₚ]_{q}—R₃ (I)

in which:

R₁ represents a saturated or unsaturated, linear or branched hydrocarbon-based chain containing from 7 to 30 carbon atoms,

R₂ represents, independently of R₁, a hydrogen atom or a saturated or unsaturated, linear or branched hydrocarbon-based chain containing from 7 to 30 carbon atoms,

R₃ represents, independently of R₁ or R₂, a hydrogen atom or a saturated or unsaturated, linear or branched hydrocarbon-based chain containing from 1 to 30 carbon atoms,

m, n, p and q are, independently of each other, equal to 0 or 1, it being understood that when R₃ represents a hydrogen atom, q is other than 0.

In formula (I) as defined above, R₁, R₂ and R₃ generally represent, independently of each other, a radical chosen from heptyl, octyl, nonyl, decyl, undecyl, dodecyl, tridecyl, tetradecyl, pentadecyl, hexadecyl, heptadecyl, octadecyl, nonadecyl, eicosyl, uneicosyl, docosyl, heptadecenyl, eicosenyl, uneicosenyl, docosenyl, heptadecadienyl and decenyl radicals.

The group R₁—C(═O)— more particularly represents an octanoyl (caprylyl), decanoyl, undecylenoyl, dodecanoyl (lauroyl), tetradecanoyl (myristyl), hexadecanoyl (palmitoyl), octadecanoyl (stearyl), eicosanoyl (arachidoyl), docosanoyl (behenoyl), 8-octadecenoyl (oleyl), eicosenoyl (gadoloyl), 13-docosenoyl (erucyl), 9,12-octadecadienoyl (linoleoyl) or 9,12,15-octadecatrienoyl (linolenoyl) radical.

Examples of compounds of formula (I) as defined above more particularly include the compounds of formula (Ia):

R₁—(C═O)—O—CH₂—CH[O—[C(═O)]ₘ—R₂]—CH₂—O—[C(═O)]ₚ—R₃ (Ia)

corresponding to formula (I) as defined above in which q and n are equal to 1, or a mixture of compounds of formula (Ia) and preferably,

either a compound of formula (Ia₁):

R₁—(C═O)—O—CH₂—CH(OH)—CH₂—OH (Ia₁)

corresponding to formula (Ia) as defined above, in which m and p are equal to 0 and R₂ and R₃ represent a hydrogen atom,

or a compound of formula (Ia₂):

R₁—(C═O)—O—CH₂—CH(OH)—CH₂—O—C—(═O)—R₃ (Ia₂)

corresponding to formula (Ia) as defined above in which p is equal to 1, m is equal to 0 and R₂ represents a hydrogen atom,

or a compound of formula (Ia₃):

R₁—(C═O)—O—CH₂—CH[O—C(═O)—R₂]—CH₂—O—C(═O)—R₃ (Ia₃)

corresponding to formula (Ia) as defined above in which m and p are equal to 1,

or a mixture of compounds of formulae (Ia₁), (Ia₂) and/or (Ia₃).

Examples of compounds of formula (Ia₁), (Ia₂) or (Ia₃) include fatty acid triglycerides or mixtures of fatty acids such as the mixture of triglycerides of fatty acids containing from 6 to 10 carbon atoms, sold under the name Softenol™ 3819, the mixture of triglycerides of fatty acids containing from 8 to 10 carbon atoms, sold under the name Softenol™ 3108, the mixture of triglycerides of fatty acids containing from 8 to 18 carbon atoms, sold under the name Softenol™ 3178, the mixture of triglycerides of fatty acids containing from 12 to 18 carbon atoms, sold under the name Softenol™ 3100, the mixture of triglycerides of fatty acids containing 7 carbon atoms, sold under the name Softenol™ 3107, the mixture of triglycerides of fatty acids containing 14 carbon atoms, sold under the name Softenol™ 3114, or the mixture of trigylcerides of fatty acids containing 18 carbon atoms, sold under the name Softenol™ 3118, glyceryl dilaurate, glyceryl dioleate, glyceryl isostearate, glyceryl distearate, glyceryl monolaurate, glyceryl monooleate, glyceryl monoisostearate and glyceryl monostearate or a mixture of these compounds.

Other examples of compounds of formula (I) as defined above include the compounds of formula (Ib):

R₁—(C═O)—O—R₃ (Ib)

corresponding to formula (I) as defined above in which q is equal to 0, or a mixture of compounds of formula (Ib).

Examples of compounds of formula (Ib) include isopropyl myristate, octyldodecyl myristate, octyl palmitate, isopropyl palmitate and isononyl isononate.

In the context of the present invention, the term “effective amount” more particularly means an amount of between 0.5% and 85% by weight of the final composition and most particularly between 0.5% and 45% by weight of final composition.

A subject of the invention is more particularly a composition as defined above, characterized in that it concerns pressed powders.

According to another particular aspect of the present invention, a subject of this invention is a composition as defined above, in which the polymer is a copolymer of methyl methacrylate with one or other of the monomers chosen from butyl acrylate, 1-methylpropyl acrylate, 2-methylpropyl acrylate and 1,1-dimethylethyl acrylate, and most particularly a composition as defined above in the form of pressed powders in which the polymer used is a copolymer of methyl methacrylate and of butyl acrylate.

The compact powder obtained is supple but strong, the compacts obtained are very significantly less hard, which makes it easier to take up the powder onto the applicator and to release it onto the skin. The compounds of the invention very significantly improve the compressibility of pressed powders.

The compounds according to the invention may be used in pressed powders of all types, irrespective of the final application, and are compatible with all the cosmetic ingredients known to those skilled in the art.

This ease of compression is accompanied by an effect on the rheological and sensory properties of the formulated powder, which is superior to the effects obtained with “incompactable” fillers, i.e:

an improvement in the feel—increasing the softness of the powder when spread onto the skin

an improvement in the uniformity of the film of powder on the skin,

better distribution of the colour, and

an improvement in the adhesion of the powder to the skin.

The composition which forms the subject of the present invention is prepared by mixing the powders of starting products and then compressing the mixture obtained. An additional advantage in using the abovementioned polymers in the composition that is the subject of the present invention lies in the fact that the process does not necessarily comprise an extrusion step before the compression step.

According to another aspect of the present invention, a subject of this invention is the use of a composition as defined above, as a powder intended for enhancing the appearance of the face or the body: care or makeup powders such as powders for unifying the complexion, satiny and moisturizing powders for the body, makeup rouges, eye shadows, etc.

The examples that follow illustrate the invention without, however, limiting it.

Demonstration of the advantages of the compositions according to the invention relative to those of the prior art.

Test (I): Simplified formulations comprising 15% by weight of a hydrophobic binder, Schercemol™ 1688, 0.6% by weight of a dye (Ariabel rose) and 84.4% of a polymer powder were prepared in the following manner.

The powder is ground in a knife mill for a few seconds, the hydrophobic binder is then added and the mixture is again ground for a few seconds. The powder is then compacted in a metal dish using a Kemwall™ manual compactor at a pressure of 30×10⁵ Pa.

The visual appearance of the compact obtained is assessed under standard luminosity. Various criteria are noted:

the level of filling of the dish,

the planarity of the surface,

the friability of the compact,

the distribution of the dye.

The ease of uptake of the powder with a brush is graded on a scale of grades ranging from 0 to 5, 0 corresponding to no uptake and 5 corresponding to very easy uptake and a large amount of powder retained on the brush.

The hardness of the compacts is measured using a texturometer equipped with a hemispherical measuring probe. The greater the compression force required to drive the probe into the compact, the greater the hardness of the compact.

The polymer powders used giving a formulation of the prior art are Micropearl™ M 305, which is a poly(methyl methacrylate) (formulation A) and Nylon™-12 (formulation B).

The polymer powder used giving two formulations according to the invention, formulation 1 and formulation 2, is a powder of copolymer of methyl methacrylate and of butyl acrylate.

The results of the observations are given in the table below.

| | Formu- | Formu- | Formu- | |
|---|---|---|---|---|
| | lation A | lation B | lation 1 | Formulation 2 |
| Visual | Incomplete | Good filling; | Good | Good filling; |
| appearance | filling; | irregular | filling; | smooth |
| of the pow- | smooth | surface; | smooth | surface; a |
| der in their | surface; a | crumbly | surface; a | few |
| dish after | few | | few | irregular- |
| compression | irregular- | | irregular- | ities at the |
| | ities at | | ities at | edge |
| | the edge | | the edge | |
| Feel | Very soft | Very soft | Very soft | Very soft |
| Visual | Smooth | Compact | Smooth | Smooth |
| appearance | surface; a | disintegrated | surface; a | surface; a |
| of the com- | few | spontaneous- | few | few |
| pacts in their | irregular- | ly before | irregular- | irregular- |
| dish after | ities at the | handling | ities at the | ities at the |
| storage for | edge | | edge | edge |
| one month | | | | |
| Ease of | 0.5 | 5 | 4 | 4 |
| uptake of the | | | | |
| powder | | | | |
| Maximum | 23.3 | 9.9 | 10.6 | 9.9 |
| compression | | | | |
| force (in | | | | |
| newtons) | | | | |

These results demonstrate that the formulations according to the prior art give either compressed powders with a smooth surface but great hardness, which are virtually impossible to take up, or powders that are easy to take up but very fragile and which break spontaneously over time. In contrast the compositions according to the invention have a very different behaviour which simultaneously combines the suppleness of the compact and the perfect maintenance of its integrity over time, without adversely affecting the sensory qualities of the powder, the pressed powder obtained having good cohesion and being easy to take up with a brush.

Test (II): Simplified formulations comprising 50% by weight of Mica 1000, 5% by weight of Lanol™ 1688, 5% by weight of DC™ 200, 0.4% by weight of dye (FDC Yellow No. 6 aluminium lake), 0.2% by weight of a dye (Ariabel Sienna), 5% by weight of polymer powder and the remainder to 100% by weight of Luzenac™ 00 talc, were prepared in the following manner:

The pulverulent compounds are mixed together and then ground in a knife mill. The hydrophobic binders are then added one after the other, with grinding of the mixture between each addition. The final mixture is ground again for a few seconds. The powder is then compacted in a metal dish using a Kemwall™ manual compactor at a pressure of 30×10⁵ Pa. The visual appearance of the compacts is observed under standard luminosity.

The ease of uptake of the powder with a brush is graded on a scale from 0 to 5: 0 corresponding to no uptake and 5 corresponding to very easy uptake and a large amount of powder retained on the brush.

The hardness of the compacts is measured using a texturometer equipped with a hemispherical measuring probe. The greater the compression force required to drive the probe into the compact, the greater the hardness of the compact.

The sensory qualities of the formulation are assessed by a panel of 15 individuals, who grade the following criteria on a scale from 0 to 5:

The ease of uptake on the finger:

0=no uptake; 5=very easy uptake

The ease of spreading on the skin:

0=difficult spreading, not smooth; 5=very easy spreading

The release of the dye:

0=poor covering of the skin; 5=uniform colour

The uniformity of the film:

0=heterogeneous film; 5=totally uniform film

Distribution of the colour:

0=poorly distributed dye; 5=uniformly distributed dye

The feel on the skin:

0=coarse feel; 5=very soft feel.

The strength of the compacts is evaluated after a test of dropping from a height of 20 cm onto a Plexiglas support. The test is performed on 10 dishes, and the result is expressed as the average number of drops that causes the pressed powder to break.

The polymer powders used giving a formulation of the prior art are Micropearl™ M 305 (formulation C) and Nylon™-12 (formulation D).

The polymer powder used giving the formulations according to the invention, formulation 3 and formulation 4, is the powder of copolymer of methyl methacrylate and of butyl acrylate.

The results of the observations are given in the table below:

| | Formu- | Formu- | Formu- | Formu- |
|---|---|---|---|---|
| | lation C | lation D | lation 3 | lation 4 |
| Visual | Smooth | Smooth | Smooth | Smooth |
| appearance of | appearance | appearance | appearance | appearance |
| the compacts | | | | |
| after | | | | |
| compression | | | | |
| Ease of | 1.2 | 2 | 4 | 4 |
| uptake of the | | | | |
| powder on a | | | | |
| brush | | | | |
| Maximum | 22.6 | 23.7 | 10.6 | 11.7 |
| compression | | | | |
| force (N) | | | | |
| Ease of | 2.5 | 2.5 | 4.2 | 4.5 |
| uptake on the | | | | |
| finger | | | | |
| Ease of | 3 | 3 | 4.5 | 4.8 |
| spreading on | | | | |
| the skin | | | | |
| Release of | 2 | 2.2 | 3.8 | 4.3 |
| the dye | | | | |
| Feel on the | 3 | 3.5 | 4 | 4.8 |
| skin | | | | |
| Number of | 12 | 9 | 13 | 12 |
| drops causing | | | | |
| the compact | | | | |
| to break | | | | |

These results demonstrate the advantages obtained with the compositions according to the invention. The methyl methacrylate/butyl acrylate copolymers also make it possible to improve the sensory qualities of the formulation, and the volunteers particularly noted the improvement in the distribution of the colour and the greater adhesion on the skin of the pressed powder.

The copolymer powders as described above may also be advantageously used as texturing agents in any form of care or makeup product. Using the compounds according to the invention very significantly improves the softness on application and offers properties adhesion of on the skin that are superior to those of the usual powders containing spherical particles.

Fluid foundation for greasy skin

| | | |
|---|---|---|
| A | Aqua/water | 20.00% |
| | Butylene glycol | 4.00% |
| | PEG-400 | 4.00% |
| | Pecosil PS100 | 1.00% |
| | Sodium hydroxide | qs pH = 9 |
| | Titanium dioxide | 7.00% |
| | Talc | 2.00% |
| | Yellow iron oxide | 0.80% |
| | Red iron oxide | 0.30% |
| | Black iron oxide | 0.05% |
| B | Montanov L | 1.50% |
| | Lanol 99 | 10.00% |
| | Diisostearyl malate | 10.00% |
| C | Aqua/water | qs 100% |
| | Tetrasodium EDTA | 0.05% |
| | Powder of copolymer of methyl | 5.00% |
| | methacrylate and of butyl acrylate | |
| D | Simulgel NS | 2.00% |
| E | Sepicontrol A5 | 4.00% |
| F | Sepicide HB | 0.30% |
| | Sepicide CI | 0.20% |
| | Perfume/fragrance | 0.20% |

Procedure

Mix together the liquid compounds of phase A and then adjust the pH to 9 approximately before adding the pigments. Grind this pigmentary phase with a ball mill (prepare an amount greater than that theoretically required on account of the losses).

Melt phase B at 80-85° C.

Heat the water to 75° C. in the main tank and then add the Micropearl M305, the EDTA and the pigmentary paste A, with continued heating. Next, introduce the fatty phase (B); next, switch on the emulsifying machine (rotor-stator) and switch off the heating. Add the Sepicontrol A5—leave the emulsifying machine running for a few minutes at 3000 rpm (depending on the amount manufactured) and then introduce the Simulgel NS and switch the emulsifying machine on again for a few minutes. Allow the mixture to cool gradually with moderate stirring and add the constituents of F at about 30° C. Adjust the final pH if necessary.

In presentation forms involving a dispersion of powders and being in solid form at the end, the compounds according to the invention contribute towards the moulding operations and prevent the phenomenon of splitting frequently observed over time with powder texturing agents containing spherical particles, in particular for high working doses.

Healthy-complexion cast powder

| | | |
|---|---|---|
| A | Talc | 10.00% |
| | Mica | 20.00% |
| | Ariabel Rose | 0.80% |
| B | Cyclomethicone | 20.00% |
| | Powder of copolymer of methyl | 12.00% |
| | methacrylate and of butyl acrylate | |
| C | Lanol 99 | qs 100% |
| | Propylene glycol | 15.00% |
| | Ozokerite | 7.00% |
| | Sepifeel One | 5.00% |

Procedure

Grind phase A with a knife mill

Mix B, add B to A with stirring—heat the mixture to 50° C

Add molten C with stirring

Cast the mixture and cool.

Lipstick

| | | |
|---|---|---|
| A | Diisopropyl dilinoleate dimer | 22.50% |
| | Titanium dioxide | 6.44% |
| | Yellow iron oxide | 3.04% |
| | Black iron oxide | 0.36% |
| | DC Red 7 | 0.78% |
| | DC Red 27 | 0.00% |
| | FDC Yellow 6 | 0.70% |
| | FDC Blue 1 | 0.17% |
| B | Diisopropyl dilinoleate dimer | 10.70% |
| | Ozokerite | 11.75% |
| | Pentaerythrityl tetralaurate | 10.00% |
| | Octyldodecanol | 8.12% |
| | Beeswax | 6.20% |
| | Triisostearyl trilinoleate | 5.00% |
| | Cetyl palmitate | 4.50% |
| | Carnauba wax | 2.28% |
| | Powder of copolymer of methyl | 5.00% |
| | methacrylate and butyl acrylate | |
| C | Sepilift DPHP | 1.00% |
| | Fragrance | 1.25% |
| | Tocopheryl acetate | 0.20% |

Procedure:

Grind the pigmentary paste using a Dispermat

Heat phase A and B on a water bath at 90° C.

Gradually introduce the waxes as they melt

Mix together the two molten phases for 10 minutes using an anchor stirrer

Introduce the ingredients of C and mix for 3 minutes using an anchor stirrer

Cast the paste into the moulds.

The characteristics of the products used in the preceding examples are as follows:

Sepicide™ CI, imidazolinurea, is a preserving agent sold by the company SEPPIC.

Sepicide™ HB, which is a mixture of phenoxyethanol, methylparaben, ethylparaben, propylparaben and butylparaben, is a preserving agent sold by the company SEPPIC.

Pecolsil™ PS100 is dimethicone copolyol phosphate sold by the company Phoenix.

Lanol™ 99 is isononyl isononanoate sold by the company SEPPIC.

Sepifeel™ One is a mixture of palmitoylproline, magnesium palmitoyl glutamate and magnesium palmitoyl sarcosinate, such as those described in FR 2 787 323.

Sepicontrol™ A5 is a mixture of capryloyl glycine, sarcosine and extract of Cinnamon zylanicum sold by the company SEPPIC.

Simulgel™ NS is a thickening reverse latex as described in international patent application WO 99/36445.

1. Use of a polymer or a blend of polymers chosen from copolymers or terpolymers of methyl methacrylate with one or more monomers chosen from butyl acrylate, 1-methylpropyl acrylate, 2-methylpropyl acrylate, 1,1-dimethylethyl acrylate, butyl methacrylate, 1-methylpropyl methacrylate, 2-methylpropyl methacrylate and 1,1-dimethylethyl methacrylate, as texturing agent in cosmetic or pharmaceutical compositions formulated in powder form, which may be applied to the human or animal body.

2. Use as defined in claim 1, in which the cosmetic or pharmaceutical compositions are pressed powders.

3. Use as defined in either of claims 1 and 2, of a copolymer of methyl methacrylate with one or other of the monomers chosen from butyl acrylate, 1-methylpropyl acrylate, 2-methylpropyl acrylate and 1,1-dimethylethyl acrylate.

4. Use as defined in claim 3, of a copolymer of methyl methacrylate and of butyl acrylate.

5. Cosmetic or pharmaceutical composition formulated in the form of a powder, which may be applied to the human or animal body, characterized in that it comprises:
an effective amount of a polymer or of a blend of polymers as defined in one of claims 1 to 4, and
at least one cosmetically acceptable compound.

6. Composition as defined in claim 5, characterized in that it is pressed powders.

7. Composition as defined in either of claims 5 and 6, in which the polymer is a copolymer of methyl methacrylate with one or other of the monomers chosen from butyl acrylate, 1-methylpropyl acrylate, 2-methylpropyl acrylate and 1,1-dimethylethyl acrylate.

8. Composition as defined in claim 6, in which the polymer used is a copolymer of methyl methacrylate and of butyl acrylate.

9. Use of a composition as defined in one of claims 4 to 8, as a powder intended for enhancing the appearance of the face or the body: care or makeup powders such as powders for unifying the complexion, satiny and moisturizing powders for the body, makeup rouges or eye shadows.
